(19) United States
(12) Patent Application Publication
(10) Pub. No.: US 2023/0399678 A1
(43) Pub. Date: Dec. 14, 2023

(54) A METHOD FOR SINGLE-CELL NASCENT RNA LABELING AND SEQUENCING

(71) Applicant: SINGLERON (NANJING) BIOTECHNOLOGIES, LTD., Nanjing (CN)

(72) Inventors: Tongtong SUN, Nanjing (CN); Guoxin TANG, Nanjing (CN); Ji LI, Nanjing (CN); Jiameng SUN, Nanjing (CN)

(21) Appl. No.: 18/251,147

(22) PCT Filed: Sep. 29, 2020
(86) PCT No.: PCT/CN2020/118795

§ 371 Date: Apr. 28, 2023

(51) Int. Cl. C12Q 1/6806

(52) U.S. Cl. CPC C12Q 1/6806

(57) ABSTRACT

The present disclosure provides a sequencing method for labeling a mass of newly born single cell mRNAs, including a method for labeling a mass of newly born single cell mRNAs (Dynamic-seq technology) and a method for preparing a SCOPE single-cell sequencing library. The present disclosure uses S⁴U to mark new RNA in single cells in large quantities, cleaves single cells and captures mRNA, and constructs the obtained total RNA in transcriptomic library after IAA treatment, so as to obtain the transcriptomic sequencing library of labeled single cells.

### RELATED APPLICATIONS

The present application is a U.S. national phase application under 35 U.S.C. § 371 of International Application No. PCT/CN2020/118795, filed on Sep. 29, 2020 and published as WO 2022/067495 A1 on Apr. 7, 2022; the content of which is incorporated herein by reference in its entirety.

### TECHNICAL FIELD

The present disclosure relates to the sequencing field of RNA metabolic markers, in particular to the Dynamic-seq technology applicable to single cell sequencing and new mRNAs in mass labeled cells.

### BACKGROUND

After an mRNA is fully processed and exported to the cytoplasm, the life-span of the transcript impacts its contribution to gene expression. In fact, the stability of mRNA is thought to contribute 20-50% to overall gene regulation^{[1-3]}. mRNA decay is a highly regulated process influenced by both cis- and trans-acting factors^{[4-5]}. The accurate and reproducible measurement of mRNA decay rates is critical to elucidating how this network of factors influences RNA stability.

Gene expression is modulated at multiple stages including transcription and processing of nascent transcripts, regulation of translation efficiency and intracellular localization, and control of the rate of RNA degradation. Many questions in RNA biology involve the kinetics of RNA abundance and the effect of various players on RNA synthesis and degradation, rather than the steady-state abundance of RNA alone. Steady state measurements of RNA abundance obtained from RNA-seq approaches are not able to separate the effects of transcription from those of RNA decay in the overall abundance of any given transcript, instead only giving information on the (presumed steady-state) abundances of transcripts^{[6]}.

With the rapid development of RNA-seq technology, Single-cell RNA-seq (scRNA-seq) also emerged and replaced the traditional single-cell microarray^{[7]}. And scRNA-seq achieves gene expression profiling at single-cell resolution^{[8-9]}. With these unique capabilities, scRNA-seq has been used to dissect molecular processes in cell differentiation and to trace cell lineages in development. It is also used to analyze the cells in a lesion of disease to identify the cell types and molecular dynamics implicated in the injury^{[10]}.

High-throughput RNA sequencing (RNA-seq) and the combination of metabolic markers have become important means to study RNA, which is helpful to determine the relevant parameters of RNA synthesis and degradation in cells under various conditions, and to understand the relationship between mRNA stability and gene expression. The most widely used approach involves metabolic labeling with thiol-labeled nucleoside analogs such as S⁴U (S⁴U -tagging)^{[10]}. Metabolic labeling is a useful tool to understand RNA dynamics without perturbing RNA transcription or processing. 4-thiouridine (S⁴U) is a convenient nucleoside for metabolic labeling because it is cell-permeable, incorporated into newly transcribed RNA^{[1]}.

At present, S⁴U metabolic marker technology has been developed into various RNA metabolic marker sequencing technologies to facilitate real-time observation of cell transcription, such as SLAM-seq, NASC-seq and TimeLapse-seq^{[12-14]}. SLAM-seq is the most widely used transcriptome technology for RNA metabolism labeling. The method is used the primary thiol-reactive compound iodoacetamide (IAA), which covalently attaches a carboxy amidomethyl group to S⁴U by nucleophilic substitution. Point mutations (T-to-C transitions) observed In RNA-seq data that chemically transform S⁴U residues into cytosine analogues. SLAM-seq combines metabolic RNA labeling technology and high-throughput RNA sequencing method, which can quickly obtain gene expression dynamics in total RNA. Both the length of time and the efficiency of labeling affect the estimated rate of RNA degradation. The presence of non-informative background RNA creates additional noise to the measurements and wastes sequencing capacity. SLAM-seq is widely used in bulk mixed cell samples, which is helpful to grasp the changes of mRNA in real time. However, S⁴U labeled RNA could not be isolated from a single cell for sequencing and transcriptional activity between different cells could not be compared. However, the current application of SLA1vi-seq in single-cell sequencing is limited to a small sample size, so it is impossible to compare and analyze a large number of single cells^{[12]}.

The scSLAM-seq was developed by the University of Wuerzburg. This approach uses S⁴U alkylation for RNA metabolic markers and single-cell transcriptome sequencing techniques to directly record transcriptional activity by distinguishing between old and new RNA from thousands of genes in each cell. Developed Bayesian algorithm (Grand-SLAM) for accurate analysis of new RNA and decay rates, Grand-SLAM 2.0 can be used to synchronously analyze hundreds of single-cell SLAM-seq libraries. This method can only process hundreds of single cells, but cannot satisfy the mass single cell mRNA. metabolic marker transcriptome sequencing^{[15]}.

### SUMMARY

In this disclosure, using Singleron Independent developed SCOPE single-cell sequencing and RNA metabolic markers technologies, we developed a single cell sequencing method (scDynamic-seq) suitable for massive single-cell dynamic-seq, which can provide new ideas for studying the changes of RNA half-life and stability, identifying target gene changes mediated by RNA modification, drug screening and other aspects

### BRIEF DESCRIPTION OF DRAWINGS

FIG. 1: Schematic diagram of the present disclosure.

FIG. 2: Schematic diagram of the embodiment of the present disclosure where Mass single cell metabolic marker sequencing.

### DETAILED DESCRIPTION

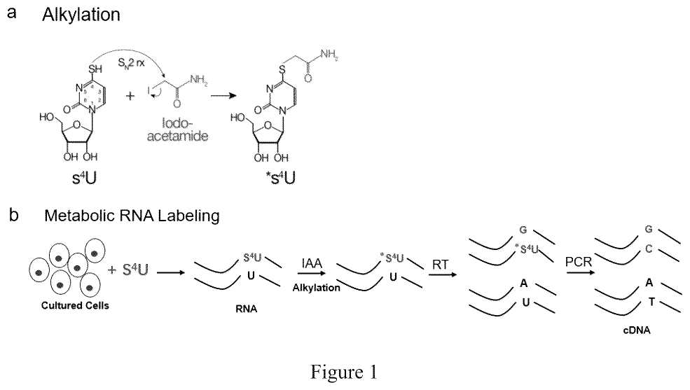

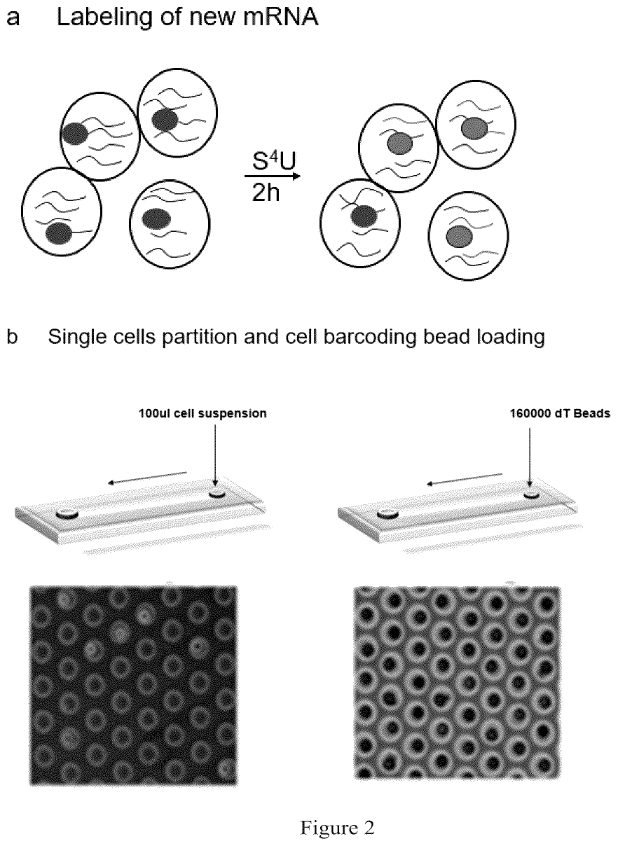

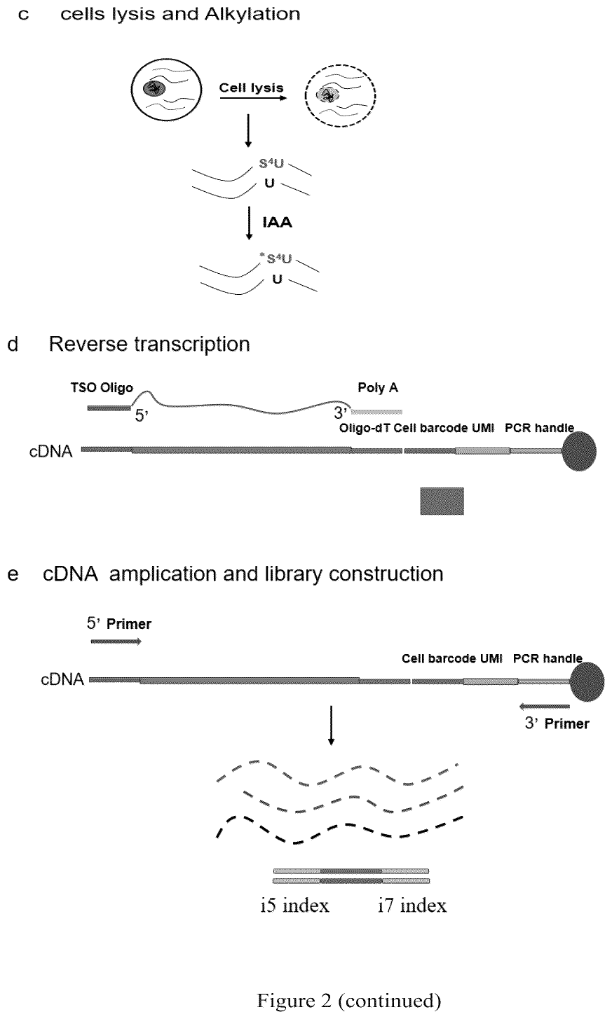

In order to apply RNA metabolic marker technology to mass single-cell sequencing, S⁴U metabolic marker was combined with innovative SCOPE single-cell sequencing preparation technology based on microfluidic chip. The cells were first metabolized with a nucleoside analogue, 4-thiuridine (S⁴U), to separate the newly synthesized RNA from the previously existing RNA, and then treated with Iodoacetamide (IAA) to convert the S⁴U -modified U bases into cytosine analogues. In reverse transcription, cytosine analogues are misidentified as C, resulting in the mismatch of G from the base A in cDNA. After sequencing, by analyzing these mismatched G, the information of the newly synthesized mRNA can be obtained (FIG. 1).

The GEXSCOPE RNA-seq library construction kit (Singleron Biotechnologies) was used to demonstrate the feasibility of the technology and the efficacy of the currently disclosed massive single-cell mRNA metabolic marker transcriptome sequencing. The experiment was carried out according to the manufacturer's requirements, and the modification is as follows.

Simply, scDynam-seq data of bone marrow of mice were dissociation. After tissue dissociation, the cells were cultured in S⁴U medium for 2 h. The single-cell suspension was loaded onto the chip and the single-cell was divided into a single hole on the chip. Two samples were prepared: one was sequenced with GEXSCOPE standard protocol for single-cell mRNA (“control”), and the other was sequenced with massive single-cell mRNA metabolic marker transcriptome (scDynamic-seq). Cell barcoding magnetic beads previously processed with PBS are loaded into microchips to wash off excess beads. Each cell bar code magnetic bead contains oligonucleotides that bind unique cell bar code sequences on the surface. Each oligonucleotide in the bead also has a unique sequence of molecular indices (UMI). The number of UMIs detected in the sequence can be used to accurately quantify different RNA molecules. Depending on the diameter of the bead and the diameter of the hole, only one bead per hole on the microchip can fall into it. Put 100 ul lysis buffer into the chip and incubate at room temperature for 20 minutes to lyse the cells. After cell lysis, add 200 ul 0.02% SSC-Tween to the chip magnet and wash away the lysis buffer. The chip was then placed on ice and injected with 100 ul of pre-cooled IAA reagent. In time, 200 ul of FC-40 was slowly injected into the chip.

| Components | Volume/Reaction (ul) |
|---|---|
| 10 mM IAA | 1 |
| 3*SSC | 49 |
| DMSO | 50 |
| Total | 100 ul |

The chips were incubated at 50° C. for 15 minutes, then placed at room temperature and on ice for 10 minutes respectively. The magnetic beads together with captured RNAs, were taken out of the microchip and subject to reverse transcription, template switching, cDNA amplification, and sequencing library construction using reagents from the GEXSCOPE kit and following manufacturer's instructions. The resulting single cell RNA-seq library was sequenced on Illumina NovaSeq with PE150 mode and analyzed with scope Tools bioinformatics workflow (Singleron Biotechnologies). Finally, the mutation frequency in the samples was used to analyze the labeling efficiency and transcriptional activity.

As shown in Table 1, Compared with the control group, the mutation frequency of the test group was significantly increased, indicating that S⁴U successfully realized. the marker on each single cell. Different marker ratios (mutation frequency) indicate different subsets of cells/different genes with different transcriptional activity, in which the higher the mutation frequency means the higher the transcriptional activity.

TABLE 1

| | | total_ | snp_ | snp_ | total_ | snp_ | snp_ | |
|---|---|---|---|---|---|---|---|---|
| Sample | cluster | cell | cell | cell % | transcript | transcript | transcript % | cell |
| Marrow | 5 | 87 | 87 | 100.00% | 176225 | 2417 | 1.37% | Mature_B_cell/plasma_ |
| | | | | | | | | cells |
| | 4 | 105 | 105 | 100.00% | 309682 | 3173 | 1.02% | Conventional type_2_ |
| | | | | | | | | dendritic_cells |
| | 1 | 177 | 175 | 98.87% | 433452 | 1582 | 0.36% | Erythroid_progenitor_ |
| | | | | | | | | cells |
| Marrow_ | 1 | 257 | 257 | 100.00% | 187774 | 57917 | 30.84% | Mature_B_cell/plasma_ |
| S4U | | | | | | | | cells |
| | 5 | 75 | 75 | 100.00% | 69244 | 19067 | 27.54% | Conventional_type_2_ |
| | | | | | | | | dendritic_cells |
| | 3 | 172 | 170 | 98.84% | 329837 | 1371 | 0.42% | Erythroid_progenitor_ |
| | | | | | | | | cells |

### REFERENCES

[1] Brenner S, Johnson M, Bridgham J, et al. Gene expression analysis by massively parallel signature sequencing (MPSS) on microbead arrays[J]. Nature Biotechnol.2000; 18(6):630-634.
[2] Peiffer J A, Kaushik S, Sakai H, et al. A spatial dissection of the Arabidopsis floral transcriptome by MPSS[J]. BMC Plant Biology, 8,1(2008-04-21), 2008, 8(1):43.
[3] Reinartz, J. Massively parallel signature sequencing (MPSS) as a tool for in-depth quantitative gene expression profiling in all organisms[J]. Brief Funct Genomic Proteomic, 2002, 1(1):95-104.
[4] Miles W O, Tschop K, Herr A, et al. Pumilio facilitates miRNA regulation of the E2F3 oncogene[J]. Genes Dev, 2012, 26(4):356-368.
[5] George R, Nahas, Raghav G, et al. The RNA-binding protein Musashi 1 stabilizes the oncotachykinin 1 mRNA in breast cancer cells to promote cell growth[J]. FASEB journal: official publication of the Federation of American Societies for Experimental Biology, 2016.
[6] Wolfe M B, Goldstrohm A C, Freddolino P L. Global analysis of RNA metabolism using bio-orthogonal labeling coupled with next-generation RNA sequencing[J]. Methods, 2018.
[7] Wang Z, Gerstein M, Snyder M. RNA-Seq: a revolutionary tool for transcriptomics[J]. Nature reviews. Genetics. 2009; 10:57-63.
[8] Tang F, Barbacioru C, Wang Y, et al. mRNA-Seq whole-transcriptome analysis of a single cell[J]. Nature methods. 2009; 6:377-382.
[9] Tang F Barbacioru C, Nordman E, et al. RNA-Seq analysis to capture the transcriptome landscape of a single cell[J]. Nature Protocols, 2010, 5(3):516.
[10] Ye Y, Song H, Zhang J, et al. Understanding the Biology and Pathogenesis of the Kidney by Single-Cell Transcriptomic Analysis[J]. Kidney Dis (Basel). 2018; 4(4):214-225.
[11] Duffy E E, Simon M D. Enriching s4U-RNA Using Methane Thiosulfonate (MTS) Chemistry[J]. Current Protocols in Chemical Biology, 2016, 8.
[12] Veronika, A, Herzog, et al. Thiol-linked alkylation of RNA to assess expression dynamics [j] . Nature Methods, 2017.
[13] Hendriks G J, Jung L A, Larsson A J M, et al. NASC-seq monitors RNA synthesis in single cells[J]. Nature communications. 2019; 10(1):3138.
[14] Schofield J A, Duffy E E, Kiefer L, et al. TimeLapse-seq: adding a temporal dimension to RNA sequencing through nucleoside recoding[J]. Nature Methods, 2018.
[15] Erhard F, Baptista M A P, Krammer T, et al. scSLAM-seq reveals core features of transcription dynamics in single cells[J]. Nature, 2019, 571(7765).

1. A sequencing method for mass labeling of newly born mRNA of single cells, comprising:
a step of labeling newly born mRNA of mass single cells; and a step of preparation of SCOPE single-cell sequencing library.

2. The method of claim 1, wherein Dynamic-seqc technique is used for labeling the newly born mRNA of mass single cells, which includes the following steps:
a. briefly exposing cells were to a medium supplemented with a nucleoside analogue, 4-thiuridine (S⁴U), which is incorporated into the new RNA during transcription;
b. loading the cells and magnetic beads into a chip, lysing the cells and capturing the mRNA; and
c. after capture, converting the S⁴U modified U base into cytosine analogue by IAA treatment.

3. The method of claim 2, wherein the nucleoside analogue 4-thiuridine (S⁴U) is used at a concentration of 100-500 uM and labeled for 0.5-24 hours.

4. The method of claim 3, wherein after mRNA capture, the total RNA is pretreated with an IAA reagent to induce alkylation of 4-mercapto groups before standard RNA-seq step.

5. The method of claim 4, wherein the IAA reagent includes: 50% DMSO, 10 mM iodoacetamide, saline sodium citrate buffer pH 8.

5. The method of claim 5, wherein the IAA reagent is added to the chip in ice, and 200 ul FC-40 is added to the chip in time.

6. The method of claim 6, wherein the chip is placed at room temperature and on ice for 10 minutes respectively after 15 min of reaction at 50° C., and 200 ul FC-40 is added into the chip to remove the bubbles.
